(19) United States
(12) Patent Application Publication
(10) Pub. No.: US 2014/0288509 A1
(43) Pub. Date: Sep. 25, 2014

(54) SYRINGE FOR A HIGH-PRESSURE INJECTOR

(71) Applicant: Medtron AG, Saarbrücken (DE)

(72) Inventors: Hanno Altmeyer, Neunkirchen (DE); Gotthilt Mehner, Sulzbach (DE)

(21) Appl. No.: 14/353,254

(22) PCT Filed: Oct. 16, 2012
(86) PCT No.: PCT/EP2012/070514

§ 371 Date: Apr. 21, 2014

(30) Foreign Application Priority Data
Oct. 19, 2011 (DE) 102011054605.7

(51) Int. Cl. A61M 5/315

(52) U.S. Cl. CPC A61M 5/31513; A61M 2005/3123
USPC 604/222

(57) ABSTRACT

A syringe for a high-pressure injector, having a syringe cylinder with a tube connection for a tube line located at an end closer to the patient and having a plunger that is guided inside the syringe cylinder in an advancing direction and that has sealing surfaces for resting in a sealed fashion against an inner wall of the syringe cylinder. The plunger is embodied as a two-component injection-molded part based on thermoplastic plastic with a core piece composed of a harder plastic and a covering layer that is mounted on the core piece and is composed of a comparatively softer material. The covering layer constitutes or forms at least the sealing surfaces and the plunger surface of the plunger oriented toward the end of the syringe cylinder closer to the patient. The plunger is embodied with or has openings in the region of its plunger surface.

### BACKGROUND OF THE INVENTION

1. Field of the Invention

This invention relates to a syringe for a high-pressure injector, having a syringe cylinder with a tube connection for a tube line located at an end closer to the patient and having a plunger that is guided inside the syringe cylinder in an advancing direction and that has sealing surfaces for resting in a sealed fashion against an inner wall of the syringe cylinder.

2. Discussion of Related Art

Syringes of this kind are used to hold a liquid to be administered to the patient, such as a contrast agent, and are inserted into an injector that then injects this liquid into the patient, such as during an imaging examination procedure. An example of such an imaging examination procedure is angiography, which requires high-pressure injectors that inject the prepared liquid, such as a contrast agent, from the syringe provided for this and into the patient at a pressure of up to 1200 PSI or 83 bar.

The syringes for high-pressure injectors used before now have a plunger guided in the syringe cylinder that is made of a hard and correspondingly dimensionally stable plastic onto which a soft, flexible plastic cap, which has at least two raised sealing rings formed onto it or at least two sealing rings inserted into it in order to resist the high working pressures of up to 83 bar during a high-pressure injection without leaking.

German Patent Reference DE 100 06 560 A1 discloses a plunger stopper composed of 2 components, an inner component that is composed of a harder material and a covering layer composed of a softer material that is integrally joined to the inner component and produces a seal in the syringe cylinder.

In addition to a good seal, considerable attention is also given to preventing improper use of such syringes, particularly to prevent such an improper use from resulting in the injection of a volume of air into a patient, which can lead to life-threatening complications.

### SUMMARY OF THE INVENTION

One object of this invention is to provide a syringe for a high-pressure injector of the type mentioned at the beginning, which can be efficiently produced, that ensures a reliable seal even at the high injection pressures that can occur, modulates the friction between the plunger and the syringe cylinder as a function of the pressure and therefore determines the input power of the injector as a function of the pressure, and offers a high level of safety in preventing injections of air due to improper use.

To attain the above and other objects, this invention includes embodying a syringe according to the features of embodiments and modifications of this invention as discussed in this specification and in the claims.

The plunger of the syringe according to this invention is embodied as a two-component injection-molded part based on thermoplastic plastic with a core piece comprising a harder plastic and a covering layer that is mounted on the core piece and that comprises a comparatively softer material that is thermally attached to the harder plastic of the core piece so that the plunger can be simply and efficiently produced using the known two-component injection molding method and can suitably cooperate with conventional syringe cylinders. The covering layer forms at least the sealing surfaces of the plunger and the plunger surface of the plunger oriented toward the end of the syringe cylinder closer to the patient. In order to absorb air volumes possibly contained in the syringe, the plunger has openings situated in the region of its plunger surface.

Such a syringe for a high-pressure injector with a plunger formed as a two-component injection-molded part with a harder core piece and a comparatively softer covering layer can be simply and inexpensively manufactured, offers a high level of functional safety, and, for example during sterilization with ethylene oxide, is easier to wet with the sterilizing agent and can therefore be sterilized in a shorter amount of contact time.

With proper use of a syringe according to this invention, a tube line of a definite length and a definite cross-section is first connected to the tube connection and the syringe is then drawn to fill it with the liquid that is to be subsequently injected, such as a contrast agent. As the syringe is drawn, first the air volume from the tube line travels into the interior of the syringe cylinder, followed by the drawn liquid. For this reason, a syringe that has been drawn in this way is first vented by sliding the plunger in the advancing direction until the air volume contained has been expelled before the tube line is connected to the patient and the injection is carried out. If, however, due to improper use, this venting of the syringe is omitted, there is the danger of injecting the volume of air into the patient.

In one embodiment according to this invention, this danger is averted by the openings provided in the plunger surface because even when the plunger has been pushed all the way in the advancing direction, these openings constitute a residual volume inside the syringe cylinder in which the existing air volume remains and for this reason, cannot be injected into the patient. In this context, this invention makes use of the fact that the syringes in a high-pressure injector are usually oriented in a downward-inclined position of the tube connection, such as 120°, so that air contained in the syringe cylinder rises to the plunger.

Since usually, only tube lines that are especially associated with such syringes are used, their maximum inner volume is known and this invention proposes embodying the openings in the plunger so that their total volume corresponds to or exceeds the inner volume of the tube line.

According to one embodiment of this invention, the sealing surfaces have at least one sealing lip that can be placed against the inner wall of the syringe cylinder.

According to another embodiment of this invention, two sealing lips are provided, with one sealing lip protruding in the advancing direction and the other sealing lip protruding in the opposite direction. This achieves an even higher degree of leak tightness in the desired pressure range of the syringe of up to 83 bar, while simultaneously improving the traveling properties of the plunger. The sealing lip protruding in the advancing direction is therefore oriented toward the hydraulic side of the plunger and the sealing lip protruding in the opposite direction is oriented toward its pneumatic side.

According to another embodiment of this invention, one sealing lip is embodied as protruding in the advancing direction and is spaced apart from the remaining body of the plunger by a free space that is open in the advancing direction, while another sealing lip is embodied as protruding in the direction opposite from the advancing direction and is spaced apart from the remaining body of the plunger by a free space that is open in the direction opposite from the advancing direction.

This arrangement of the sealing lips ensures that with increasing injection pressure of the injector, these sealing lips exert a correspondingly increasing surface pressure against the inner wall of the syringe cylinder and therefore offer reliable protection against leakage even at the expected high pressures during use in a high-pressure injector.

According to one embodiment of this invention, the plunger surface oriented in the advancing direction and a conical tip of the plunger are formed by the covering layer, thus ensuring a particularly simple manufacture. In this embodiment, the covering layer thus covers all of the surfaces of the plunger or core piece thereof that come into contact with the liquid with which the syringe is to be filled.

If two sealing lips are used, according to one embodiment of this invention, they are spaced apart from each other by a groove in the direction opposite from the advancing direction, thus ensuring a particularly smooth sliding of the plunger inside the syringe cylinder without the risk of a tilting and the accompanying weakening of the sealing function of the sealing lip. If liquid unexpectedly slips past a sealing lip, it is captured in the groove that serves as a catch basin in this case and is prevented from escaping from between the plunger and syringe cylinder.

According to one embodiment of this invention, the core piece of the plunger can be manufactured of a suitable hard polycarbonate or a possibly reinforced acryl-butadiene-styrene.

According to one embodiment of this invention, the covering layer is of a thermoplastic elastomer that can be adhesively attached to the core piece and that has a shore hardness of 60-90 A. Examples of a thermoplastic elastomer for the covering layer that can he adhesively attached to the core piece of polycarbonate or acryl-butadiene-styrene include, for example, include PP/EPDM-based thermoplastic elastomers. For example, partially cross-linked EPDM/PP blends that are sold under the trade names Sarlink® by the Teknor Apex Company or Badaprene® by Bada AG can be used for the covering layer. Possible alternatives also include polyurethanes of an appropriate hardness.

Furthermore, a coupling adapter for connection to the plunger rod of an injector can be formed onto the core piece.

Finally, toward its end oriented away from the advancing direction, the core piece can he provided with or have a base plate that can be, for example, of polycarbonate or acryl-butadiene-styrene. The base plate serves to axially stabilize the core piece and also to absorb pressure for the plunger rod of the injector.

### BRIEF DESCRIPTION OF THE DRAWINGS

Additional embodiments and particulars of this invention are explained in greater detail below in view of exemplary embodiments shown on the drawings, wherein:

FIG. 1 shows a longitudinal section taken through a syringe according to this invention;

FIG. 2 shows a section D-D taken through the syringe according to FIG. 1;

FIG. 3a schematically depicts a section taken through a first embodiment of a plunger of the syringe according to this invention, taken along the line A-A in FIG. 3b;

FIG. 3b shows a top view of the plunger according to FIG. 3a;

FIG. 4a schematically depicts a half section taken through a second embodiment of a plunger of the syringe according to this invention, taken along the line B-B in FIG. 4b; and

FIG. 4b shows a top view of the plunger according to FIG. 4a.

### DETAILED DESCRIPTION OF THE INVENTION

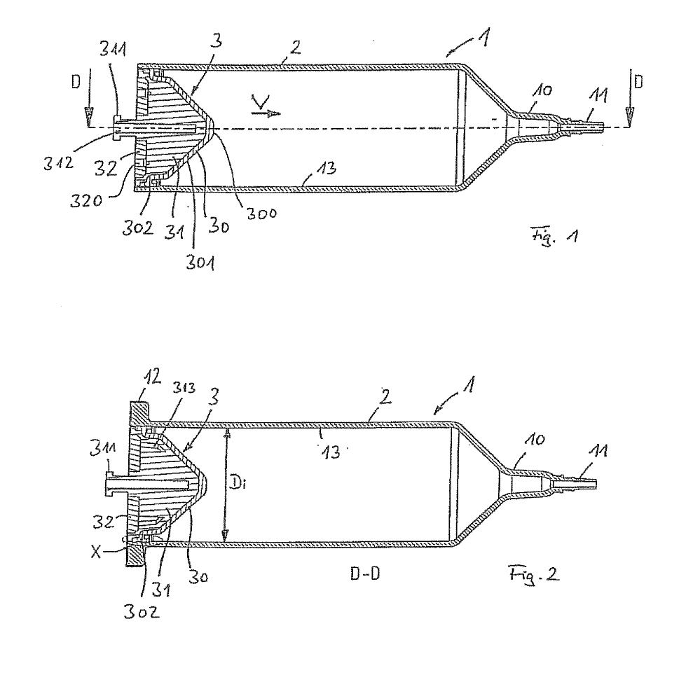

FIGS. 1 and 2 show a syringe 1 for a high-pressure injector, which is used as an accessory for a high-pressure injector and serves to prepare a liquid that is to be administered to a patient, such as a contrast agent for an imaging examination procedure.

In a known way, the syringe 1 includes a syringe cylinder 2, which can be manufactured for example of PETG or polycarbonate or can also be produced from POM with a lubricant incorporated into the matrix. The syringe cylinder 2 is usually transparent.

Inside the syringe cylinder 2, a plunger 3 is guided so that it is able to slide in a sliding and/or advancing direction V for purposes of injecting the liquid from the syringe cylinder 2. The plunger 3 can naturally also be moved in the opposite direction in order to draw liquid into the syringe. In this respect, the maximum diameter of the plunger 3 corresponds to the inner diameter Di of the syringe cylinder 2.

At its end 10 closer to or near the patient, the syringe cylinder 2 has a tube connection 11 for a tube line, not shown here, to the patient, while the plunger 3 has a coupling adapter 311 for connecting to the plunger rod, not shown here, of the injector. In addition, the flange projections 12 at the back end of the syringe cylinder 2, see FIG. 2, are used for attachment to the injector.

The plunger 3 is embodied in the form of a two-component injection-molded part based on thermoplastic plastics and includes a core piece 31 composed of or comprising a harder plastic, such as polycarbonate or acryl-butadiene-styrene (ABS), which can possibly be reinforced. Using the two-component injection molding method, this core material 31 can be adhesively covered with a suitable softer plastic material such as a thermoplastic elastomer or a polyurethane with a lubricant incorporated into the matrix and with a shore hardness of 60-90 A that simultaneously covers all of the surfaces of the plunger 3 that come into contact with the liquid that can be stored within the syringe cylinder 2, which surfaces will be described in greater detail below.

The plunger has a tip 300 with a surrounding conical surface 301 that terminates at a sealing surface 302 that rests in a sealed fashion against the inner wall 13 of the syringe cylinder 2. The tip 300, the plunger surface 301, and the adjoining sealing surface 302 are integrally composed of or comprise the softer covering layer 30.

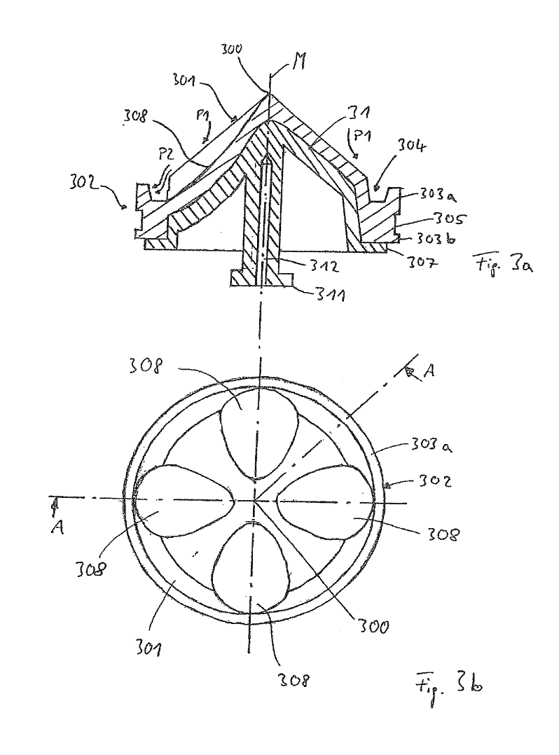

More details of the design of the sealing surface 302 are also shown in the schematic depiction according to FIG. 3a.

Starting from the plunger surface 301, the sealing surface 302 has a sealing lip 303a oriented in the advancing direction V and the sealing lip 303a is separated from the rest of the plunger surface 301 by a groove-like free space 304 that is open in the advancing direction V. In the direction opposite from the advancing direction V, the sealing lip 303a is first adjoined by an annular groove 305 and then an additional sealing lip 303b as well as a shoulder 307. In some exemplary embodiments, the sealing lips 303a and 303b share a common diameter relative to the central axis M, which corresponds to the inner diameter Di of the syringe cylinder 2, while the annular groove 305 and the shoulder 307 likewise share a common diameter, which is smaller than the diameter of the sealing lips 303a, 303b, relative to the central axis M.

With proper advancing of the plunger 3 inside the syringe cylinder 2, at the high working pressures of up to 83 bar of a high-pressure injector used for this purpose, corresponding opposing forces of the liquid being displaced out from the end 10 closer to the patient act on the plunger 3 and its sealing lip 303a, which action is indicated by the arrows P1 in the schematic depiction according to FIG. 3a. Because of the shape of the plunger surface 301 that tapers conically to a tip 300, liquid therefore travels into the free space 304 that is open in the advancing direction V, see arrow P2, and produces an increase in the surface pressure of the sealing lip 303a against the inner wall 13 of the syringe cylinder 2 in the region F, which increase is proportional to the injection pressure, so that a reliable seal is ensured in this region even at extremely high pressures. If liquid should nevertheless slip past the sealing lip 303a, it is collected in the annular groove 305 situated behind this sealing lip and prevented from escaping from the annular groove 305 past the sealing lip 303b. The sealing lip 303b also provides a precise axial guidance of the plunger along the inner wall 13 of the syringe cylinder 2.

The drawing, for example filling, of a syringe of this kind with the liquid to be injected generally occurs by a tube line that is connected to the tube connection 11 and later produces a connection to the patient. In this regard, during the drawing of the syringe, the air contained in the tube line is first drawn into the inside of the syringe cylinder 2 before being followed by the liquid. This air must then be removed by advancing the plunger 3 a limited distance in the advancing direction in order to prevent an injection of air into the patient. If this venting is forgotten, a situation can arise that is extremely dangerous for the patient.

In order to counteract this danger, the plunger surface 301 of the plunger is embodied with a plurality of, in this embodiment four, openings 308 in the plunger surface 301, which are offset from one another by 90 degrees and whose volumes relative to the imaginary rotational surface of the plunger correspond to at least the volume of the lumen of the tube line. When the plunger 3 is advanced to its farthest position in the advancing direction V, an air volume that is present in the syringe cylinder 2, for example due to the failure to perform a venting, can be completely accommodated inside the openings 308 and is reliably prevented from being discharged from the syringe into the patient. As a result, the syringe depicted achieves an extremely high level of protection from improper use.

It is also clear that the plunger 3, in the region of its core piece 31, is provided with or has cavities 313 for an improved load bearing capacity and for a reduced cycle time in the manufacture of the plunger 3. In addition, the coupling adapter 311 is formed onto the rear end and is provided with a hole 312.

To improve the load-bearing capacity of the plunger rod, not shown here, that engages the coupling adapter 311, a base plate comprising or composed of polycarbonate or ABS is inserted into the core piece 31 and secured in position by screws, for example.

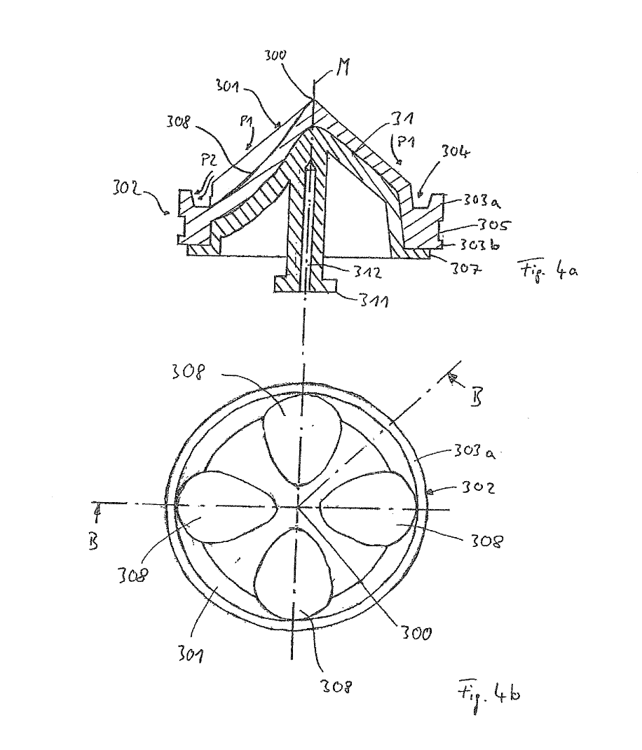

FIGS. 4a and 4b show a shape of the plunger 3 that has been modified in relation to the exemplary embodiment shown above. The same parts have been labeled with the same reference numerals and in order to avoid repetition, are not explained again wherever this is not required for comprehension of this invention.

By contrast with the exemplary embodiment according to FIGS. 3a and 3b, the plunger 3 has a second sealing lip 303b, which is embodied as protruding in the direction opposite from the advancing direction V and likewise separated from the remaining body of the plunger 3 by a free space 304b, as is also the case with the sealing lip 303a protruding in the advancing direction V and its free space 304a. This yields better properties of the plunger 3 when it is acted on by pressure and improves its traveling properties. The first sealing lip 303a is consequently oriented toward the hydraulic side of the plunger and the second sealing lip 303b is oriented toward its pneumatic side. The first sealing lip 303a mainly functions when the plunger is moving in the advancing direction V, while the second seal 303b primarily functions when the plunger is moving in the opposite direction.

Naturally, the dimensions and geometric properties of the syringe cylinder 2 and the plunger 3 that can be inferred from the drawings are merely intended to serve as examples and depend on the injector used and the area of use.

In any case, it is possible according to this invention to produce syringes that are safe to use and leak-tight even when used in high-pressure injectors at injection pressures of up to 1200 PSI, which corresponds to 83 bar.

1. A syringe for a high-pressure injector, having a syringe cylinder (2) with a tube connection (11) for a tube line located at an end (10) closer to a patient and having a plunger (3) is guided inside the syringe cylinder (2) in an advancing direction (V) and having sealing surfaces (302) for resting in a sealed fashion against an inner wall (13) of the syringe cylinder (2), the syringe comprising the plunger (3) formed as a two-component injection-molded part with a thermoplastic plastic with a core piece (31) of a harder plastic and a covering layer (30) that is mounted on the core piece (31) and of a comparatively softer material; the covering layer (30) forming at least the sealing surfaces (302) and the plunger surface (301) of the plunger (3) oriented toward the end (10) of the syringe cylinder closer to the patient; and the plunger (3) having openings (308) near a plunger surface (301).

2. The syringe according to claim 1, wherein a volume of the openings (308) in the plunger surface (301) at least corresponds to a second volume of the tube line that can be connected to the tube connection (11).

3. The syringe according to claim 2, wherein the sealing surfaces (302) include at least one sealing lip (303) that can be placed against the inner wall (13) of the syringe cylinder (2).

4. The syringe according to claim 3, wherein two sealing lips (303a, 303b) are spaced apart from each other by a groove (305).

5. The syringe according to claim 4, wherein a sealing lip (303a) protrudes in the advancing direction (V) and is spaced apart from the remaining body of the plunger (3) by a free space (304a) that is open in the advancing direction (V), and another sealing lip 303b protrudes in a direction opposite from the advancing direction (V) and is spaced apart from a remaining body of the plunger (3) by a free space (304b) that is open in the direction opposite from the advancing direction (V).

6. The syringe according to claim 5, wherein the core piece (31) of the plunger (3) is manufactured of polycarbonate or acryl-butadiene-styrene.

7. The syringe according to claim 6, wherein the covering layer (30) is of a thermoplastic elastomer that can be adhesively attached to the core piece (31) and has a shore hardness of 60-90 A.

8. The syringe according to claim 7, wherein a coupling adapter (311) for a plunger rod of an injector is formed onto the core piece (31).

9. The syringe according to claim 8, wherein the core piece (31) has a base plate (32) at an end oriented away from the advancing direction (V).

10. The syringe according to claim 1, wherein the sealing surfaces (302) include at least one sealing lip (303) that can be placed against the inner wall (13) of the syringe cylinder (2).

11. The syringe according to claim 10, wherein two sealing lips (303a, 303b) are spaced apart from each other by a groove (305).

12. The syringe according to claim 1, wherein a sealing lip (303a) protrudes in the advancing direction (V) and is spaced apart from the remaining body of the plunger (3) by a free space (304a) that is open in the advancing direction (V), and another sealing lip 303b protrudes in a direction opposite from the advancing direction (V) and is spaced apart from a remaining body of the plunger (3) by a free space (304b) that is open in the direction opposite from the advancing direction (V).

13. The syringe according to claim 1, wherein the core piece (31) of the plunger (3) is manufactured of polycarbonate or acryl-butadiene-styrene.

14. The syringe according to claim 1, wherein the covering layer (30) is of a thermoplastic elastomer that can be adhesively attached to the core piece (31) and has a shore hardness of 60-90 A.

15. The syringe according to claim 1, wherein a coupling adapter (311) for a plunger rod of an injector is formed onto the core piece (31).

16. The syringe according to claim 1, wherein the core piece (31) has a base plate (32) at an end oriented away from the advancing direction (V).
